(19) United States
(12) Patent Application Publication
(10) Pub. No.: US 2013/0319410 A1
(43) Pub. Date: Dec. 5, 2013

(54) INHALATION DEVICE, SYSTEMS, AND METHODS FOR ADMINISTERING POWDERED MEDICAMENTS TO MECHANICALLY VENTILATED SUBJECTS

(71) Applicant: THE UNIVERSITY OF KANSAS, Lawrence, KS (US)

(72) Inventors: Cory Berkland, Kansas City, KS (US); Warangkana Pornputtapitak, Kanchanburi (TH); Parthiban Selvam, Commonwealth (SG); Nashwa El-Gendy, Lawrence, KS (US)

(73) Assignee: THE UNIVERSITY OF KANSAS, Lawrence, KS (US)

(21) Appl. No.: 13/906,215

(22) Filed: May 30, 2013

Related U.S. Application Data
(60) Provisional application No. 61/653,364, filed on May 30, 2012.

(51) Int. Cl. A61M 16/10; A61M 16/04; A61M 16/20; A61M 15/00; A61M 16/00

(52) U.S. Cl. CPC A61M 16/10; A61M 15/00; A61M 16/0057; A61M 16/20; A61M 16/0434
USPC 128/203.15; 128/203.12

(57) ABSTRACT

Inhalation devices, systems, and methods for the administration of powdered medicaments to mechanically ventilated subjects are provided. In one embodiment, an inhalation device adaptively connected at one end to an air source and at the other end is operatively disposed to a ventilator circuit is provided. The inhalation devices are capable of causing a powdered medicament within a container held by the device to be dispensed from the container into the lungs of a mechanically ventilated subject.

### CROSS REFERENCE TO RELATED APPLICATIONS

This application claims the benefit of U.S. Provisional Application No. 61/653,364 filed May 30, 2012, which is incorporated by reference.

### STATEMENT OF GOVERNMENT INTEREST

None.

### BACKGROUND

Mechanical ventilation is a method of mechanically assisting or replacing spontaneous breathing when patients cannot do so. One type of ventilation system employs the use of an endotracheal or tracheostomy tube secured into a patient's upper respiratory tract. Air is mechanically delivered to the patient via the tube. In many cases, mechanical ventilation is used in acute settings such as an intensive care unit for a short period of time during a serious illness. Currently, the main form of mechanical ventilation is positive pressure ventilation, which works by increasing the pressure in the patient's airway and thus forcing additional air into the lungs.

To aid in the treatment of ventilated patients, certain medicines may be delivered via inhalation to the respiratory tract of the subject. Typically when a patient's medical condition requires administration of a medicine via inhalation, the equipment generally used to administer the medicine is a nebulizer. Nebulizers work by generating a fine aerosol of liquid particles from a solution of a medicine. This aerosol may then be administered to the patient via an endotracheal tube for a ventilator. However, not all medicines can be formulated in liquid form. Additionally, the efficacy of nebulizers may also be reduced when included in ventilator circuits as the endotracheal tube acts in part as a block to aerosol deposition.

To administer a powdered medicament, a dry powder inhaler may be used. However, these devices typically rely on inspired air drawn through the unit by the patient to aerosolize the powdered medicament. Thus, these devices suffer from the problem that they require activation by the patient.

### SUMMARY

The present disclosure generally relates to inhalation devices, systems, and methods for the administration of powdered medicaments to mechanically ventilated subjects. More particularly, the present disclosure relates to inhalation devices that are operatively connected to a ventilator circuit, as well as systems and methods suitable for delivering powdered medicaments into the lungs of a mechanically ventilated subject.

The features and advantages of the present invention will be apparent to those skilled in the art. While numerous changes may be made by those skilled in the art, such changes are within the spirit of the invention.

### DRAWINGS

Some specific example embodiments of the disclosure may be understood by referring, in part, to the following description and the accompanying drawings.

FIGS. 1A and 1B depict an inhalation device of the present disclosure, according to one embodiment.

FIG. 1C are photographs of an inhalation device of the present disclosure, according to one embodiment.

FIG. 1D depict an inhalation device of the present disclosure, according to one embodiment.

FIG. 2 depicts an inhalation device of the present disclosure, according to one embodiment, in connection with an air source.

FIG. 3 depicts a system of the present disclosure comprising an inhalation device of the present disclosure, according to one embodiment, in connection with an endotracheal tube and a ventilator.

FIGS. 4A and 4B depict a Monodose inhaler alone (4A) and in connection with an air source (4B).

FIG. 5 is a graph depicting the comparative delivery efficiencies of a nanocluster formulation of a dry powder and a micronized particle formulation of a dry powder as measured using a cascade impactor and a Monodose inhaler.

FIG. 6 is a graph depicting the effect of inspiration pattern on the delivery efficiency of a dry powder as measured using a cascade impactor and Monodose inhaler.

FIG. 7 is a graph depicting the effect of volumetric flow rates on the delivery efficiency of a dry powder as measured using a cascade impactor and a modified Monodose inhaler.

FIG. 8 is a graph depicting the effect of inspiration volume on the delivery efficiency of a dry powder as measured using a cascade impactor and a modified Monodose inhaler.

FIG. 9 is a graph depicting the effect of relative humidity on the delivery efficiency of a dry powder as measured using a cascade impactor and a modified Monodose inhaler.

FIG. 10 is a graph depicting the comparative delivery efficiencies of a dry powder as measured using a cascade impactor and either an inhalation device of the present disclosure or a modified Monodose inhaler.

FIG. 11 is a graph depicting the effect of inspiration air flow source (ventilator vs. ventilator bag) on the delivery efficiency of a dry powder as measured using a cascade impactor and an inhalation device of the present disclosure.

FIG. 12 is a graph depicting the effect of inhalation time on the delivery efficiency of a dry powder as measured using a cascade impactor and an inhalation device of the present disclosure.

FIG. 13 is a graph depicting the effect of inhalation time on the delivery efficiency of a dry powder as measured using a cascade impactor and an inhalation device of the present disclosure.

FIG. 14 is a graph depicting the effect of tube diameter on the delivery efficiency of a dry powder as measured using a cascade impactor and an inhalation device of the present disclosure.

FIGS. 15A and B depicts a system of the present disclosure comprising an inhalation device of the present disclosure, according to one embodiment, in connection with an air source.

FIG. 16 depicts an inhalation device of the present disclosure comprising pins, according to one embodiment.

FIG. 17 is a graph comparing three dry powder formulations.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to inhalation devices, systems, and methods for the administration of powdered medicaments to mechanically ventilated subjects. More particularly, the present disclosure relates to inhalation devices that are operatively connected to a ventilator circuit, as well as systems and methods suitable for delivering powdered medicaments into the lungs of a mechanically ventilated subject.

Mechanically ventilated subjects routinely receive warm and humidified air, and the administration of dry powders in a humid environment can reduce the aerosol dispersion performance. The present disclosure provides systems, compositions, and methods for capsule-based dry powder delivery adapted for use with a ventilator circuit to deliver therapeutic aerosols. One advantage of the certain systems of the present disclosure is that they may provide a means to integrate a device for delivery of dry powder therapeutics with a ventilator circuit. Another advantage is the ability to maintain positive pressure to hold a patient's lungs open during administration of a dry powder therapeutic.

In general, a system of the present disclosure may comprise an air source, an inhalation device operably connected to the air source, and a dry powder therapeutic formulation disposed within the inhalation device. As used herein, the term “dry powder therapeutic formulation” refers to a composition that consists of finely dispersed solid particles that are capable of being readily dispersed in an inhalation device and subsequently inhaled by a subject so that the particles reach the lungs to permit penetration into the upper and lower airways. Thus, the powder is said to be “respirable.”

The air source may be a ventilator that is part of a ventilator circuit or the air source may be a positive pressure pump. When both a ventilator and positive pressure pump are used, a valve or other control mechanism may be used to regulate the flow of air in the ventilator circuit suitable for a particular subject. Any suitable valve may be used, for example, a solenoid valve; and any suitable mechanism may be used, for example, electronic or mechanical control between the air sources.

The inhalation device is operatively connected to the air source. The inhalation device may be operatively connected to the air source and/or the ventilator circuit through tubing and various other connectors known in the art. In some embodiment, the inhalation device is included in the ventilator circuit (e.g., used in series with a ventilator circuit). In other embodiments, the inhalation device is introduced into the ventilator circuit by means of a catheter capable of insertion into a ventilator circuit (e.g., by introduction through an endotracheal tube). In still other embodiments, the inhalation device may be used in parallel (bypassing the circuit), driven by an external air source (e.g., a positive pressure pump). For example, when connected to suction catheters similar to those used to remove debris from endotracheal tubes, the dry powder therapeutic formulation may be introduced bypassing the humid and variable environment of the ventilator circuit.

The dry powder therapeutic formulation may be disposed with in the inhalation device such that the flow of air form the air source releases the dry powder therapeutic formulation into the ventilator circuit for delivery into a subject's lungs. Accordingly, the system may further comprise an endotracheal tube, for example, an endotracheal tube with an inflation cuff for sealing the lung from backflow of air.

The dry powder therapeutic formulation may be provided by any suitable means for providing a dry powder formulation. For example, the formulation may be provided by a capsule, reservoir, or blister package.

In certain embodiments, an inhalation device of the present disclosure may comprise two pieces: a cap and a body. One end of the cap is operably connects to a catheter or to an endotracheal tube and one end of the body is designed to operably connect to the air source (e.g., a ventilator or positive pressure pump). The body of the inhalation device generally contains a receptacle into which a container comprising a powdered medicament is loaded and a cone-shaped chamber through which air passes from a ventilation source into the receptacle. As a result of the configuration of the present inhalation device, according to certain embodiments, when air is passed from the cone-shaped chamber into the receptacle, the medicament container spins within the receptacle and powdered medicament is released through holes present in the medicament container. In certain embodiments, the inhalation device may be similarly structured but be formed from one piece.

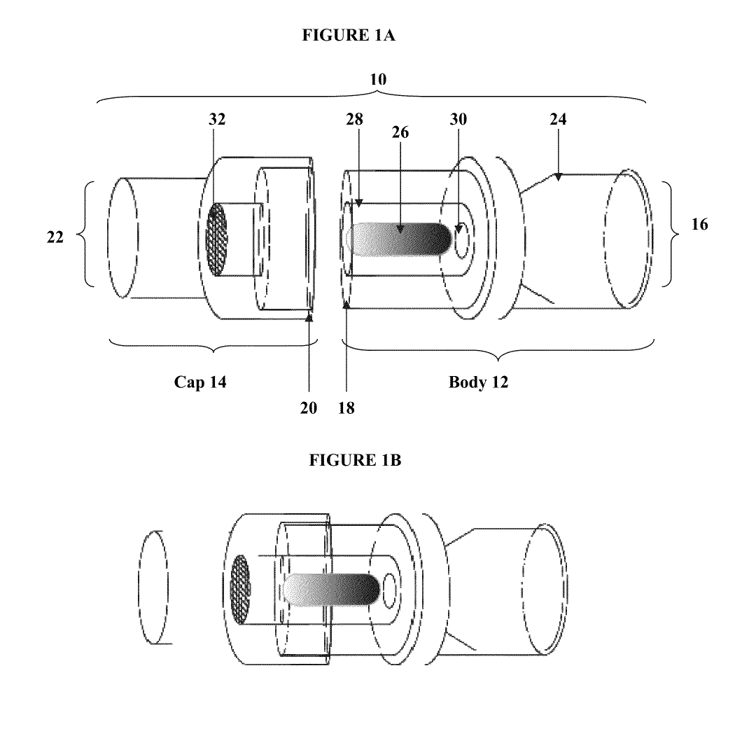

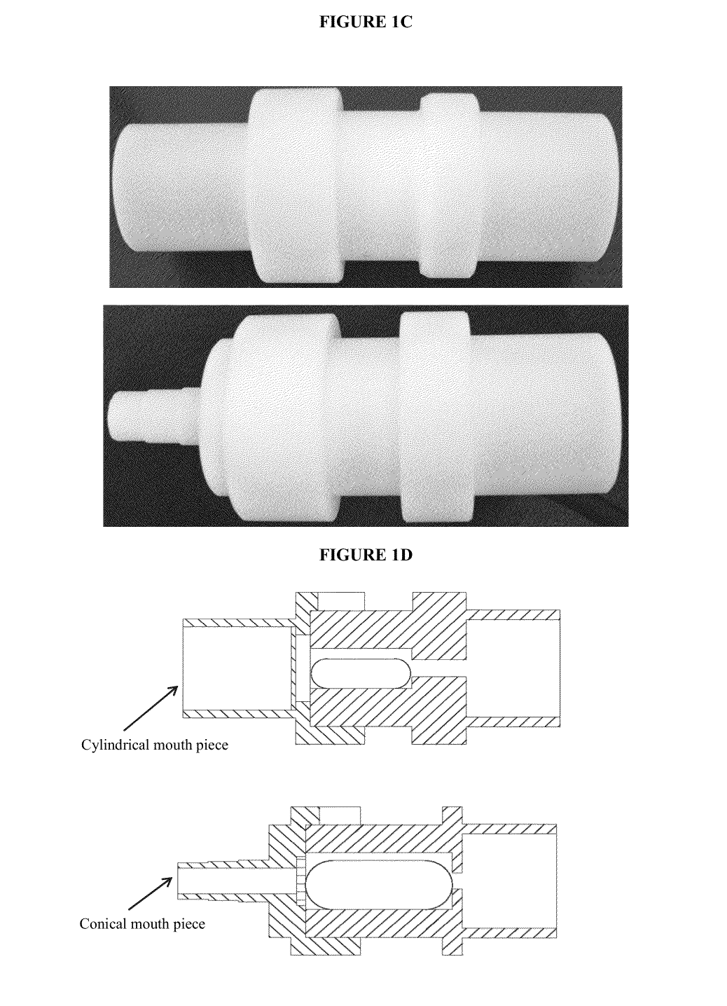

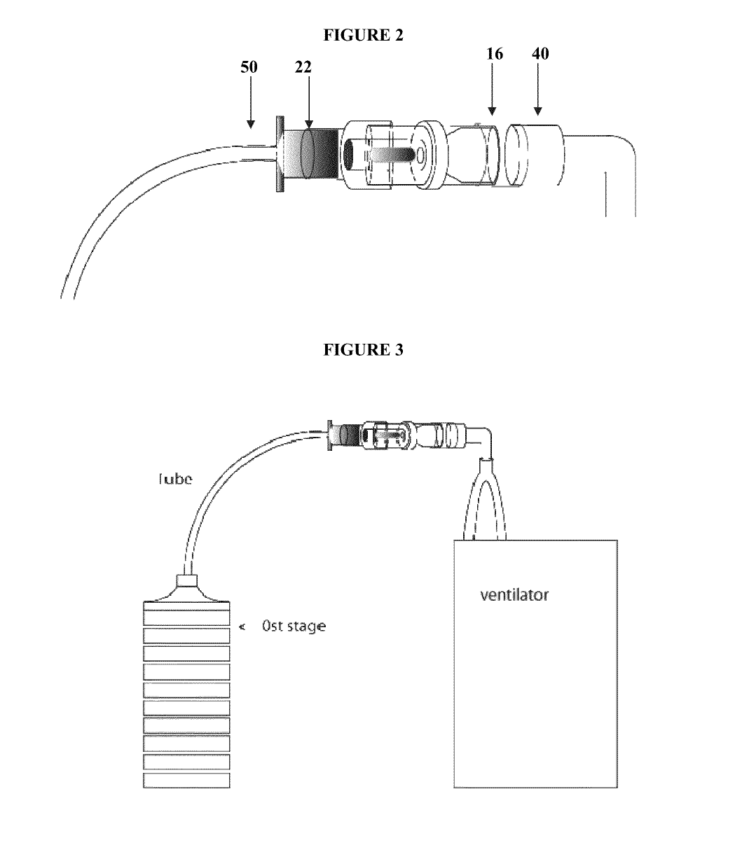

Referring first to FIG. 1A and FIG. 2, an inhalation device 10 is shown having a body 12 and a cap 14 which are adapted to fit together as shown in FIG. 2A. At one end of body 12 is an inlet 16 intended for connection with an inlet tube 40 (e.g., tubing to the air source). The other end 18 of body 12 is received by cap 14. Cap 14 likewise has one end 20 that receives body 12 and an outlet 22 at the other end intended for connection with an outlet tube 50 (e.g., endotracheal tube or catheter tube).

The body 12 further has a cone-shaped chamber 24 and a receptacle 26 which is configured to hold a medicament container 28 for the dry powder therapeutic formulation (e.g., a capsule) in such a manner so as to allow medicament container 28 to spin within receptacle 26 when air is passed from cone-shaped chamber 24 through an opening 30 into receptacle 26. Generally, chamber 24 is cone-shaped so as to reduce the resistance of air passing through the device, but other shapes may be suitable so long as the dry powder therapeutic formulation is adequately provided. In addition, receptacle 26 and opening 30 are sized so as to facilitate the spinning of medicament container 28 within receptacle 26. Once the powdered medicament is expelled from medicament container 28 it passes through an opening 32 (e.g., mesh, holes, or other discontinuous openings) in cap 14 and into outlet tube 50.

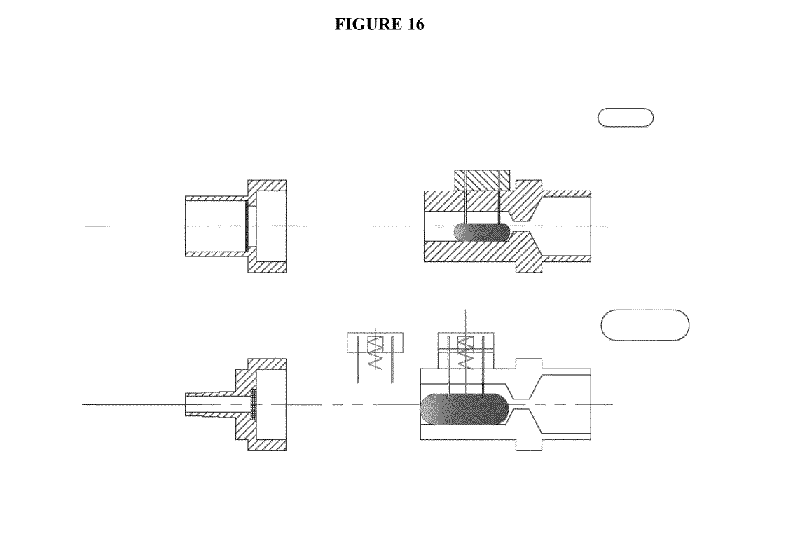

In certain embodiments, the inhalation device may comprise pins for puncturing a medicament container. For example, as shown in FIG. 16.

In operation, air from an air source (e.g., a ventilator, ventilator bag, or positive pressure pump) passes through tubing 40 into cone-shaped chamber 24 and through an opening 28 into receptacle 26. The air causes medicament container 28 to spin within receptacle 26 and the powdered medicament is expelled from holes within medicament container 28. The powdered medicament is entrained by the airstream and passes through opening 32 in cap 14 into outlet tube 50 (e.g. a catheter tube) and carried into the lungs of the user for beneficial or therapeutic action thereof to occur.

Suitable inhalation devices of the present disclosure may be made of any suitable material, including but not limited to a plastic material such as nylon, polyacetal or polypropylene, or a metal.

The physical properties of the dry powder therapeutic formulation will affect the manner in which it is dispensed from an inhalation device. However, for a given powdered medicament, varying the size or shape of chamber 24, the diameter of opening 30, and/or the size or shape of receptacle 26, inhalation devices can be made to deliver the powdered medicament in a different number of inhalations or in a longer or shorter period of time.

In general, the dry powder therapeutic formulations useful in the devices, systems, and methods of the present disclosure should have an emitted fraction appropriate for delivery into a subject's lungs. More specifically, the dry powder therapeutic formulations suitable for use in the present disclosure should have an emitted fraction greater than 60%, greater than 65%, or greater than 75% as measured by the “emitted fraction test.” The emitted fraction test, as used herein, is performed as follows: A Monodose is loaded with a capsule (HPMC type, size 3) that has been filled with 3 mg of dry powder. An Anderson Cascade Impactor (ACI) at a pro-rated flow rate of 90 L min⁻¹ is controlled using an external air source and fitted to test tubing (e.g., endotracheal tube, catheter, and the like). The cut-off aerodynamic diameter for the pre-separator is 5 μm. Before actuation, the capsule is punctured and the aerosolized powder is drawn through the ACI. After actuation, the capsule and any device components along with components of the ACI are washed with predetermined volumes of a suitable buffer (e.g., phosphate buffer pH 3.2) or solvent. Appropriate sample dilutions are performed followed by measurements with UV-Vis spectrophotometer at 280 nm or other suitable detection method. The following parameters may then be determined from the ACI dispersion data: (1) Fine Particle Dose (FPD), which is the amount of drug deposited in the filter, (2) Fine Particle Fraction (FPF), which is the percentage of drug deposited on the filter with respect to emitted dose, (3) emitted dose (ED), which is the amount of dose delivered, and (4) emitted fraction (EF), which is the percentage of emitted dose with respect to the total dose. In certain embodiments, the dry powder therapeutic formulation also may have a mass mean aerodynamic diameter less than 3.5 μm. Mass mean aerodynamic diameter may be determined, for example, using Anderson Cascade Impaction or time-of-flight measurement (TOF).

The dry powder therapeutic formulations useful in the devices, systems, and methods of the present disclosure may be in the form of nanoclusters as described in U.S. Patent Publication No. 2011/0223203, which is incorporated by reference herein. In other embodiments, suitable dry powder therapeutic formulations may be in the form of spray dried particles, according to techniques known in the art.

The present disclosure also provides, according to certain embodiments, methods for delivering a dry powder therapeutic formulation to a subject's lungs, for example, into the lungs of a mechanically ventilated subject. In such methods, the dry powder therapeutic formulation has an emitted fraction greater than 60%, greater than 65%, or greater than 75% as measured by the emitted fraction test. In certain embodiments, the dry powder therapeutic formulation also may have a mass mean aerodynamic diameter less than 3.5 μm.

To facilitate a better understanding of the present invention, the following examples of certain aspects of some embodiments are given. In no way should the following examples be read to limit, or define, the entire scope of the invention.

### EXAMPLES

### Budesonide Nanocluster Delivery Through Endotracheal Tubes or Catheters

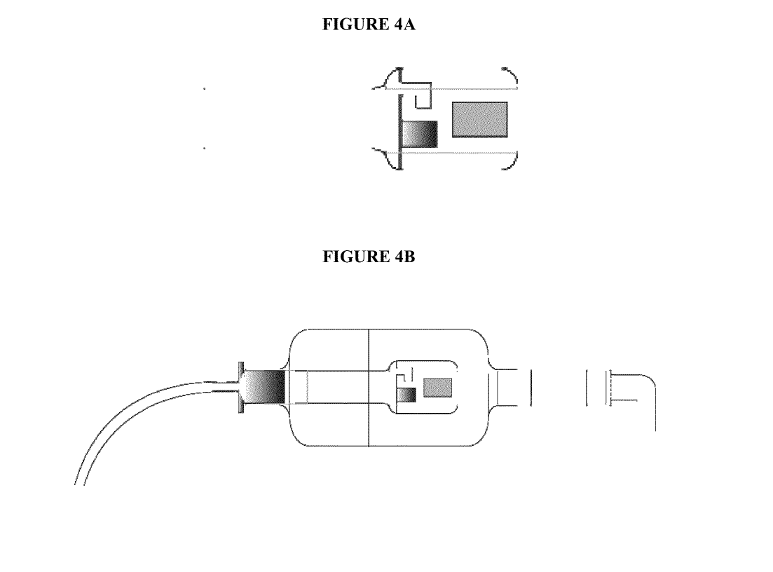

Cascade impaction was performed to determine aerosol and Monodose performance. A cascade impactor was connected to a ventilator and the Monodose (shown in FIG. 4A) was integrated as shown in FIG. 4B. The flow rate, inspiration volume, inspiration pattern, and humidity were controlled by the ventilator.

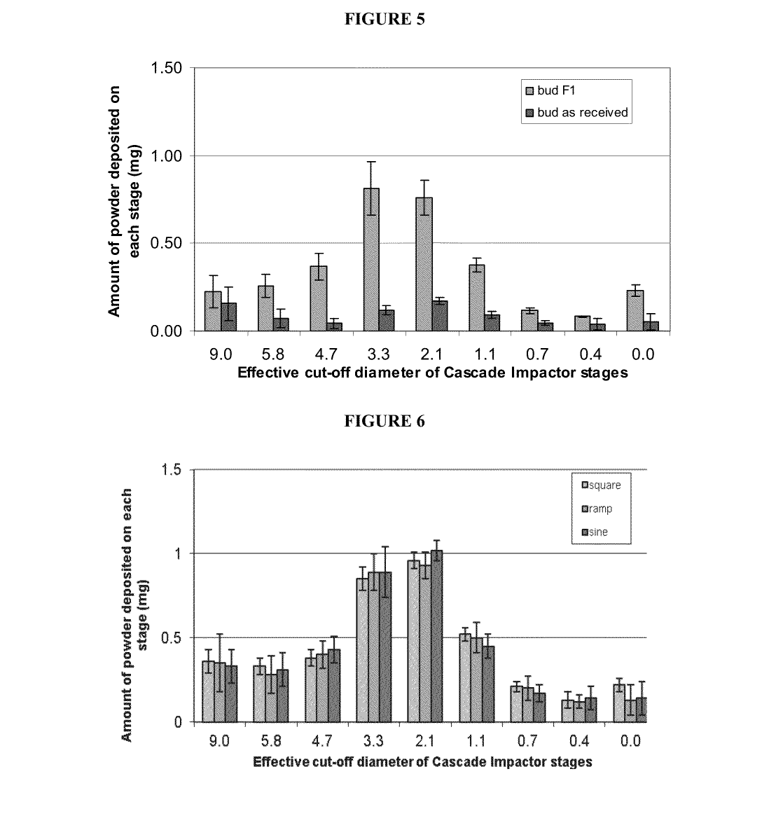

Nanocluster budesonide (NC-Bud) and budesonide as received (i.e., micronized budesonide) were applied through an endotracheal tube (5.0 mm ID). The ventilator was operated at 30 L/min. A 2.5-L inspiration volume and sine-wave-form inspiration pattern was applied. As shown in Table 1 below and FIG. 5, NC-Bud showed a percent emitted fraction (% EF) much higher than budesonide as received, although the mass median aerodynamic diameter (MMAD) was not different between NC-Bud and budesonide as received. The geometric standard deviation (GSD) of NC-Bud was 2.4±0.1, smaller than the GSD of budesonide as received (3.6±0.9).

TABLE 1

| | | % RF | | | |
|---|---|---|---|---|---|
| Formulation | % EF | <5.7 | <3.3 | MMAD | GSD |
| NC-Bud | 64.6 ± 7.3 | 85.2 ± 3.3 | 48.8 ± 6.6 | 2.2 ± 0.3 | 2.4 ± 0.1 |
| bud as received | 15.9 ± 3.3 | 72.8.6 ± 12.3 | 52.1 ± 12.7 | 2.1 ± 0.8 | 3.6 ± 0.9 |

Cascade Impaction Results of Budesonide when applying through 5.0 mm endotracheal tube (ID = 5.0 mm) (Values = Average ± SD).

Effect of Inspiration Pattern on Powder Performance

A Monodose was used to deliver NC-Bud through an endotracheal tube (ID=5.0 mm) at a flow rate of 30 L/min. Different inspiration patterns were applied. As shown in Table 2 below, the percent emitted fraction (% EF) of NC-Bud under square wave form was similar to the % EF under ramp wave form and sine wave form. The mass median aerodynamic diameter (MMAD) of NC-Bud under three different inspiration patterns was not significantly different. The geometric standard deviation (GSD) of these experiments was around 2.4 to 2.6. From this, it can be concluded that the inspiration pattern did not affect the powder performance of NC-Bud when applied through 5.0-mm endotracheal tube at flow rate of 30 L/min (FIG. 6).

TABLE 2

| Inspiration | | % FPF | | | |
|---|---|---|---|---|---|
| pattern | % EF | <5.7 | <3.3 | MMAD | GSD |
| square | 78.9 ± 3.8 | 82.6 ± 2.3 | 51.5 ± 3.6 | 2.0 ± 0.2 | 2.6 ± 0.0 |
| wave form | | | | | |
| ramp | 76.1 ± 8.2 | 83.5 ± 4.0 | 49.6 ± 1.4 | 2.1 ± 0.1 | 2.5 ± 0.2 |
| wave form | | | | | |
| sine | 77.8 ± 10.0 | 83.5 ± 3.7 | 49.7 ± 2.6 | 2.1 ± 0.1 | 2.4 ± 0.1 |
| wave form | | | | | |

Cascade Impaction Results of Budesonide when applying through endotracheal tube (ID = 5.0 mm) at flow rate of 30 L/min (Values = Average ± SD).

Effect of Flow Rate on Powder Performance

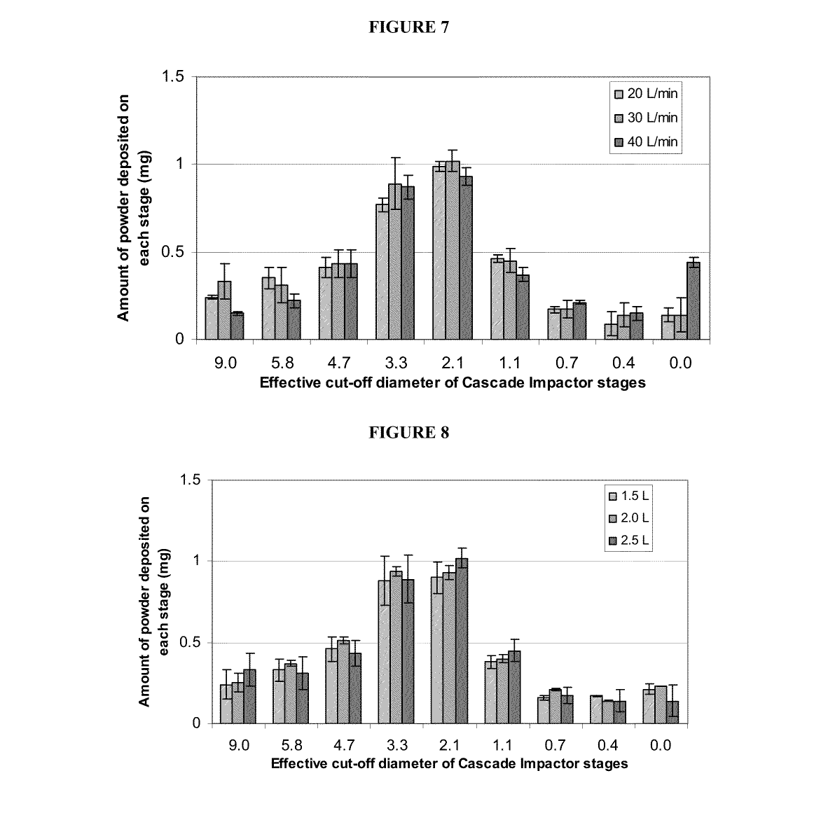

The modified Monodose was used to deliver NC-Bud through an endotracheal tube (ID=5.0 mm) at different volumetric flow rates. The 2.5-L inspiration volume and sine-wave-form inspiration pattern were applied for all experiments. However, the powder performance was not significantly different for volumetric flow rates in the range of 20-40 L/min. (Table 3, FIG. 7).

TABLE 3

| Flow rate | | % FPF | | | |
|---|---|---|---|---|---|
| (L/min) | % EF | <5.7 | <3.3 | MMAD | GSD |
| 20 | 72.4 ± 5.4 | 83.8 ± 1.0 | 51.2 ± 0.8 | 2.1 ± 0.1 | 2.4 ± 0.1 |
| 30 | 77.8 ± 10.0 | 83.5 ± 3.7 | 49.6 ± 2.6 | 2.1 ± 0.1 | 2.4 ± 0.1 |
| 40 | 75.6 ± 5.8 | 90.2 ± 0.7 | 55.8 ± 2.1 | 1.9 ± 0.1 | 3.0 ± 0.1 |

Cascade Impaction Results of Budesonide when applying through endotracheal tube (ID = 5.0 mm) at different flow rates (Values = Average ± SD).

Effect of Inspiration Volume on Powder Performance

The modified Monodose was used to deliver NC-Bud through an endotracheal tube (ID=5.0 mm) at flow rate of 30 L/min. The 2.5-L inspiration volume and sine-wave-form inspiration pattern were applied. The % EF, % FPF, MMAD and GSD were almost the same when inspiration volume of 1.5, 2.0 and 2.5 L were applied (Table 4). Therefore, the variable of inspiration volume did not affect the aerosolization of drug powder (FIG. 8).

TABLE 4

| volume | | % FPF | | | |
|---|---|---|---|---|---|
| (L) | % EF | <5.7 | <3.3 | MMAD | GSD |
| 1.5 | 74.6 ± 7.7 | 84.8 ± 2.9 | 48.8 ± 2.1 | 2.2 ± 0.1 | 2.5 ± 0.1 |
| 2.0 | 79.8 ± 3.2 | 84.3 ± 2.6 | 48.0 ± 4.0 | 2.2 ± 0.2 | 2.5 ± 0.3 |
| 2.5 | 77.8 ± 10.0 | 83.5 ± 3.7 | 49.6 ± 2.6 | 2.1 ± 0.1 | 2.4 ± 0.1 |

Cascade Impaction Results of Budesonide when applying through endotracheal tube (ID = 5.0 mm) at different inspiration volume (Values = Average ± SD).

Effect of Humidity of Inspired Air on Powder Performance

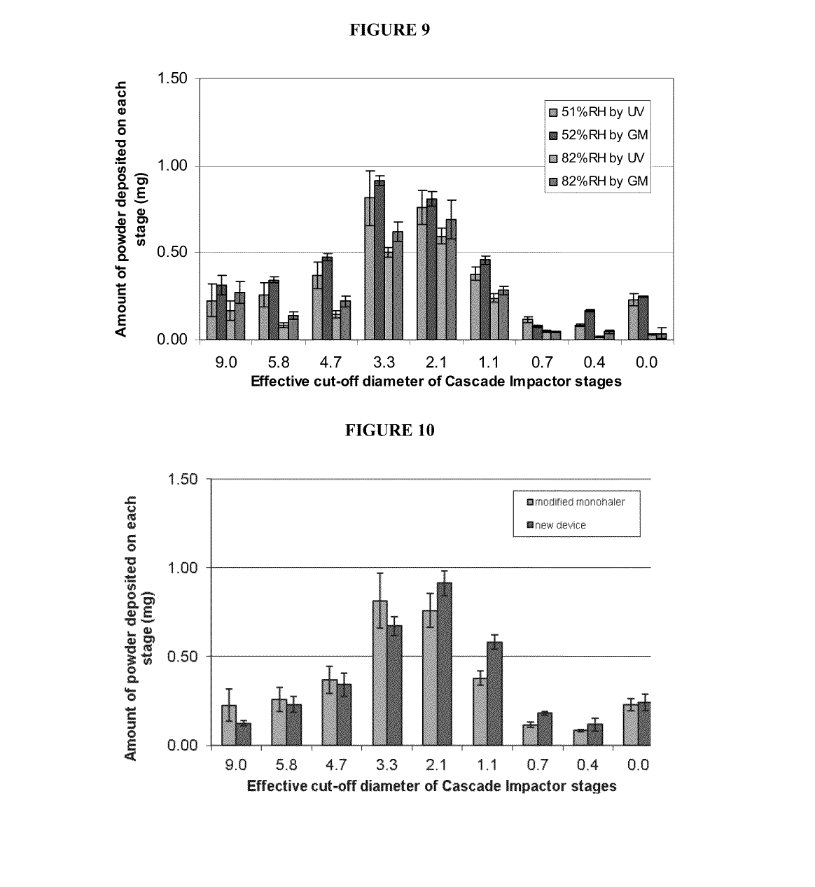

The modified Monodose was used to deliver NC-Bud through an endotracheal tube (ID=5.0 mm) at flow rate of 30 L/min. The sine wave form and inspiration volume of 2.5 L were applied for all experiments. The % EF of NC-Bud when operated at 82% RH was lower than the % EF of NC-Bud when operated at 51% RH although the distribution of aerosol powder at 82% RH shifted slightly toward smaller MMAD (Table 5, FIG. 9).

TABLE 5

| Relative | | | | | |
|---|---|---|---|---|---|
| Humidity | | % RF | | | |
| (% RH) | % EF | <5.7 | <3.3 | MMAD | GSD |

by UV

| | | | | | |
|---|---|---|---|---|---|
| 51 | 64.6 ± 7.3 | 85.2 ± 3.3 | 48.8 ± 6.6 | 2.2 ± 0.3 | 2.4 ± 0.1 |
| 82 | 36.6 ± 2.1 | 86.4 ± 3.6 | 50.8 ± 2.8 | 2.1 ± 0.1 | 2.0 ± 0.2 |

by gravimetric

| | | | | | |
|---|---|---|---|---|---|
| 51 | 76.2 ± 7.1 | 82.7 ± 1.2 | 46.4 ± 3.6 | 2.3 ± 0.2 | 2.5 ± 0.1 |
| 82 | 47.1 ± 2.6 | 82.4 ± 3.6 | 46.7 ± 3.2 | 2.2 ± 0.1 | 2.2 ± 0.2 |

Cascade Impaction Results of Budesonide when applying through endotracheal tube (ID = 5.0 mm) at different relative humidity (Values = Average ± SD).

Powder Performance on Inhalation Device of the Present Disclosure

An inhalation device of the present disclosure was used to deliver NC-Bud to the ventilator circuit and the endotracheal tube. The aerosol was applied via the inhalation device through 5.0-mm endotracheal tube at flow rate of 30 L/min. The sine wave form and inspiration volume of 2.5 L were applied. The inhalation device of the present disclosure showed higher efficiency on NC-Bud delivery as compared to the modified Monodose (i.e., the % EF of NC-Bud when applied via the inhalation device of the present disclosure was slightly higher than NC-Bud when applied via modified Monodose). The distribution of NC-Bud also shifted toward a smaller MMAD when applied via an inhalation device of the present disclosure. The GSD of both experiments was around 2.3 to 2.4. (Table 6, FIG. 10).

TABLE 6

| | | % RF | | | |
|---|---|---|---|---|---|
| Device | % EF | <5.7 | <3.3 | MMAD | GSD |
| modified | 64.6 ± 7.3 | 85.2 ± 3.3 | 48.8 ± 6.6 | 2.2 ± 0.3 | 2.4 ± 0.1 |
| Monodose | | | | | |
| inhalation | 68.0 ± 6.2 | 89.6 ± 0.7 | 60.0 ± 1.5 | 1.7 ± 0.1 | 2.3 ± 0.1 |
| device | | | | | |

Cascade Impaction Results of Budesonide when applying through endotracheal tube (ID = 5.0 mm) (Values = Average ± SD).

Powder Performance on Ventilator Bag

An inhalation device of the present disclosure was used to deliver NC-Bud through an endotracheal tube (ID=5.0 mm). A ventilator bag was applied to provide the inspiration air flow compared to the ventilation. The % EF of NC-Bud when delivered by using a ventilator was higher than when delivered by using a ventilator bag. However, the efficiency of NC-Bud delivery via ventilator bag depended on the technique used by the operator. The ventilator bag resulted in higher MMAD compared to the ventilator. The GSD of both experiments were around 2.3 to 2.4. (Table 7, FIG. 11).

TABLE 7

| | | % RF | | | |
|---|---|---|---|---|---|
| Device | % EF | <5.7 | <3.3 | MMAD | GSD |
| ventilator | 68.0 ± 6.2 | 89.6 ± 0.7 | 60.0 ± 1.5 | 1.7 ± 0.1 | 2.3 ± 0.1 |
| ventilator | 63.9 ± 0.7 | 84.4 ± 1.4 | 49.2 ± 2.5 | 2.2 ± 0.1 | 2.4 ± 0.1 |
| bag | | | | | |

Cascade Impaction Results of Budesonide when applying through endotracheal tube (ID = 5.0 mm) (Values = Average ± SD).

Effect of Inhalation Time on Powder Performance (on Ventilator Bag)

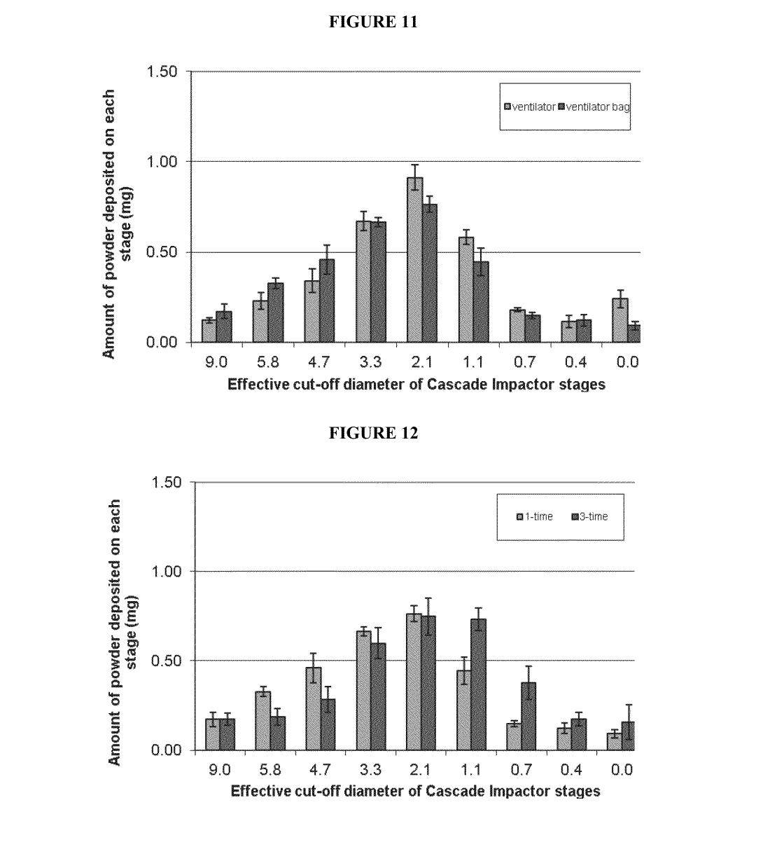

NC-Bud was applied through a 5.0-mm endotracheal tube by using an inhalation device of the present disclosure. A ventilator bag was used to provide the inspiratory air flow through the inhalation device. The flow rate depends on the operator. In this experiment, the flow rate was measure at around 23 L/min each time. Applying three cycles of inhalation showed % EF slightly higher than a single inhalation. Longer inhalation time results in shifting of the distribution toward smaller MMAD (Table 8, FIG. 12).

TABLE 8

| Inhalation | | | | | |
|---|---|---|---|---|---|
| Device ven- | | % RF | | | |
| tilator bag | % EF | <5.7 | <3.3 | MMAD | GSD |
| 1-time | 63.9 ± 0.7 | 84.4 ± 1.4 | 49.2 ± 2.5 | 2.2 ± 0.1 | 2.4 ± 0.1 |
| 3-time | 68.6 ± 9.0 | 89.3 ± 1.8 | 63.6 ± 1.8 | 1.4 ± 0.1 | 2.6 ± 0.1 |

Cascade Impaction Results of Budesonide when applying through endotracheal tube (ID = 5.0 mm) (Values = Average ± SD).

Effect of Inhalation Time on Powder Performance

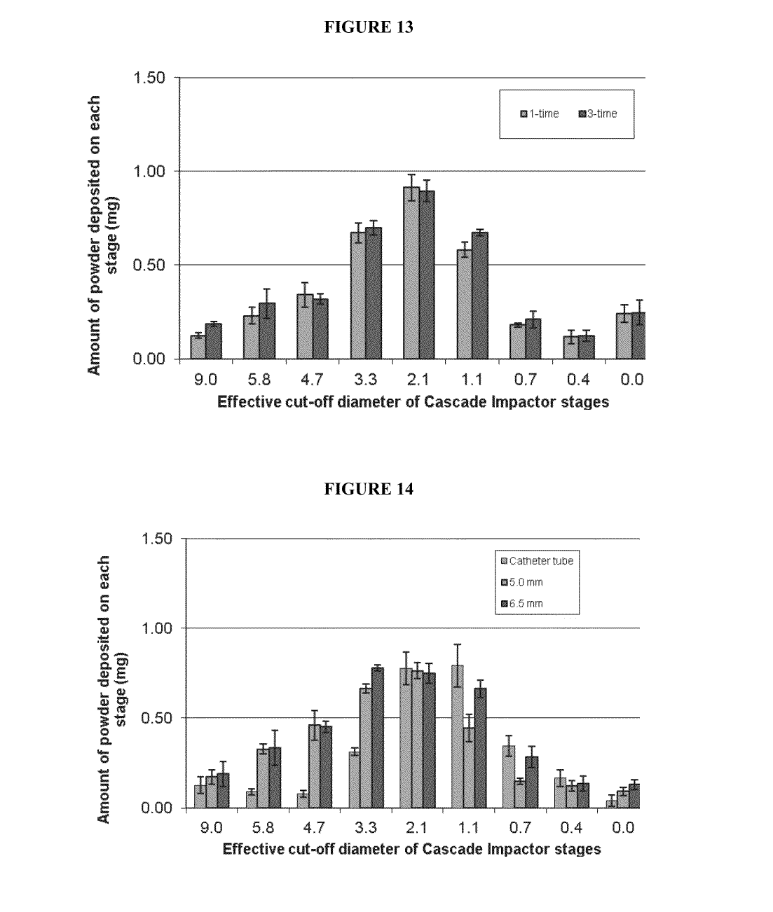

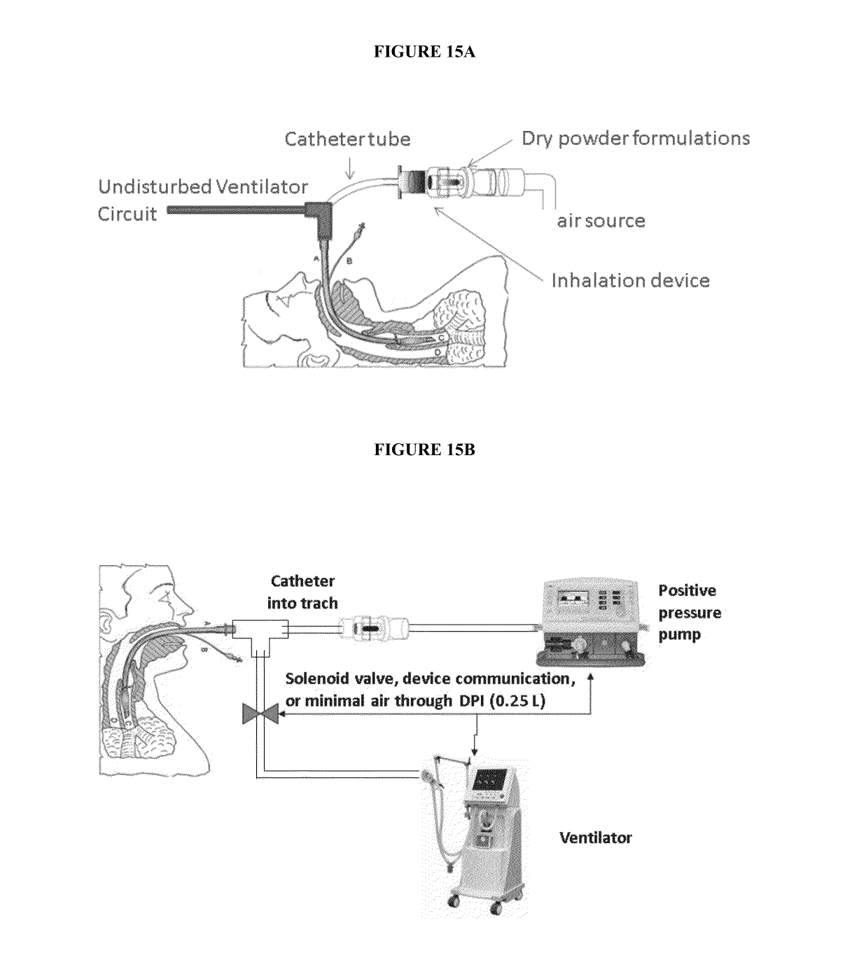

NC-Bud was applied via an inhalation device of the present disclosure through a 5.0-mm endotracheal tube. The flow rate was 30 L/min, 2.5 L inspiration volume and sine-wave-form inspiration pattern was controlled by the ventilator. Although the inhalation time would affect the powder performance when applied using a ventilator bag, not much affect was observed when using the ventilator. However, the % EF of NC-Bud when applying three cycles of inspiration was slightly higher than when applying a single inspiration. (Table 9, FIG. 13).

TABLE 9

| New inhaler | | % RF | | | |
|---|---|---|---|---|---|
| ventilator | % EF | <5.7 | <3.3 | MMAD | GSD |
| 1-time | 68.0 ± 6.2 | 89.6 ± 0.7 | 60.0 ± 1.5 | 1.7 ± 0.1 | 2.3 ± 0.1 |
| 3-time | 72.9 ± 4.7 | 86.8 ± 1.9 | 58.9 ± 2.8 | 1.7 ± 0.1 | 2.5 ± 0.1 |

Cascade Impaction Results of Budesonide when applying through endotracheal tube (ID = 5.0 mm) (Values = Average ± SD).

Effect of Tube Diameter on Powder Performance

NC-Bud was delivered by using a ventilator bag combined with an inhalation device of the present disclosure. NC-Bud was applied through different diameter tubes. The bigger diameter tube provided a higher % EF of NC-Bud. The distribution of the NC-Bud shifted toward smaller MMAD when applied through the smaller diameter tubes, especially the catheter tube (˜3 mm). The GSD of these experiments were around 2.1 to 2.5 (Table 10, FIG. 14).

TABLE 10

| Device | | % RF | | | |
|---|---|---|---|---|---|
| ven-bag | % EF | <5.7 | <3.3 | MMAD | GSD |
| Catheter tube | 54.6 ± 2.6 | 92.0 ± 2.1 | 77.6 ± 2.2 | 1.1 ± 0.0 | 2.1 ± 0.2 |
| 5.0 mm | 63.9 ± 0.7 | 84.4 ± 1.4 | 49.2 ± 2.5 | 2.2 ± 0.1 | 2.4 ± 0.1 |
| 6.5 mm | 74.3 ± 4.5 | 86.0 ± 3.1 | 52.9 ± 1.4 | 1.9 ± 0.1 | 2.5 ± 0.1 |

Cascade Impaction Results of Budesonide when applying through endotracheal tube (ID = 5.0, 6.5 mm) and catheter tube (Values = Average ± SD).

Spry-Dried Powder Formulation

A dry powder therapeutic formulation according to the present disclosure was prepared by spray drying. The resulting particles were smooth and spherical (1-2 microns in diameter) as analyzed by SEM.

The aerodynamic diameter and size distributions of the dry powders were determined by time-of-flight measurement (TOF) using an Aerosizer LD (Amherst Instruments, Hadely, Mass.) equipped with a 700 mm aperture operating at 6 psi. Approximately 1 mg of the powder was added to the instrument disperser and data were collected for ˜60 s under high shear (˜3.4 kPa). The instrument size limits were 0.10-200 μm and particle counts were above 100,000 for all measurements. The particles were in the respirable size range (2.10 mm±1.7 mm) with relatively narrow size distribution. For the drug powder as received, the mean aerodynamic diameter (MAD) was 2.84 mm±1.87 μm. This measurement, however, only included fine particles (particle count less than 10,000) and the bulk of the powder remained in the dispersing bin of the instrument. The aerodynamic particle size further indicated the transformation of the drug from poorly dispersing to a fine dispersible powder.

Aerosol Characterization by FSI

The emitted fraction percentage is determined as follows: A Monodose is loaded with a capsule (HPMC type, size 3) that has been filled with 3 mg of dry powder. A Fast Screening Impactor (FSI) at a pro-rated flow rate of 90 L min⁻¹ is controlled using an external air source and fitted to test tubing (e.g., endotracheal tube, catheter, and the like). The cut-off aerodynamic diameter for the pre-separator was 5 μm. Before actuation, the capsule is punctured and the aerosolized powder is drawn through the FSI. After actuation, the capsule and any device components along with components of the FSI are washed with predetermined volumes of a suitable buffer (e.g., phosphate buffer pH 3.2) or solvent. Appropriate sample dilutions are performed followed by measurements with UV-Vis spectrophotometer at 280 nm or other suitable detection method. The following parameters may be determined from the FSI dispersion data: (1) Fine Particle Dose (FPD), which is the amount of drug deposited in the filter, (2) Fine Particle Fraction (FPF), which is the percentage of drug deposited on the filter with respect to emitted dose, (3) emitted dose (ED), which is the amount of dose delivered, and (4) emitted fraction (EF), which is the percentage of emitted dose with respect to the total dose.

The ventilator was set to deliver an inspiratory flow volume of 2.5 L with a square wave inspiratory pattern and flow rate of 20 L/min at 25% relative humidity (RH). FSI was conducted by delivering drug as received and spray-dried drug at 60 LPM through a 3 mm ID catheter tube within an 8.5 mm ID endotracheal tube. Both dry powders had approximately the same emitted fraction (EF) of ˜73%. The spray-dried drug had a higher fine particle fraction (FPF) and fine particle dose (FPD). The FPF was around 50% which was nearly double that of the drug as received (Table 11). The superior performance of the spray-dried formulation was likely due to the smaller particle size, narrower size distribution, and particle morphology.

TABLE 11

| Aerosol parameters | Spray-dried | as received |
|---|---|---|
| FPF^{a} % (<5 um) | 50 ± 5.0 | 24 ± 0.1 |
| FPD^{b} (<5 um, mg) | 6.6 ± 0.4 | 3.6 ± 0.1 |
| ED^{c} (mg) | 13.3 ± 0.3 | 15 ± 1.0 |
| EF^{d} (%) | 73 ± 2.0 | 79 ± 3.0 |

^{a}FPF: Fine Particle Fraction
^{b}FPD: Fine Particle Dose
^{c}ED: Emitted dose
^{d}EF: Emitted fraction
FSI Spray-dried powder compared to the drug as received at 60 L/min. (n = 3; ±S.D.).

Effect of Ventilator Flow Rate

The ventilator was connected to the 8.5 mm ID endotracheal tube and set to deliver an inspiratory flow volume of 2.5 L with a square wave inspiratory flow rate at 20 or 60 L/min and 25% RH. Spray-dried powder formulation (20 mg) was delivered through a 3 mm ID catheter tube inserted within the endotracheal tube. The external air source provided 2 L of air at 60 L/min through an inhalation device connected to the catheter. The emitted dose increased by 2.3 mg for a ventilator flow rate of 20 L/min compared to 60 L/min (Table 12). The 60 L/min yielded a comparative increase in device retention. Even though both ventilator flow rates had a statistically similar FPF of ˜48%, a higher FPD was achieved for the 20 L/min flow rate owing to the increased EF %. High inspiratory flow rates may increase turbulent flow leading to inertial impaction of aerosol particles and decrease aerosol deposition during mechanical ventilation.

TABLE 12

| | 60 L/min | 20 L/min |
|---|---|---|
| | Ventilator | Ventilator |
| Aerosol parameters | flow rate | flow rate |
| FPF % (<5 um) | 49 ± 3.0 | 48 ± 2.0 |
| FPD (<5 um, mg) | 5 ± 0.3 | 5.9 ± 0.4 |
| ED (mg) | 10 ± 0.03 | 12.3 ± 0.3 |
| EF (%) | 55 ± 0.1 | 68 ± 1.0 |

FSI of spray-dried drug at different ventilator flow rates. (n = 3; ±S.D.).

Effect of Inspiratory Flow Volume

The ventilator was set to deliver a 2.5 L inspiratory flow volume with a square wave inspiratory flow of 20 L/min and 25% RH. Spray-dried powder (20 mg) was delivered using an inhalation device at 60 L/min at an inhalation volume of 1 L, 1.5 L or 2 L applied through a 3 mm ID catheter placed within the 8.5 mm ID endotracheal tube. It was clear that there was no significant difference in the FPF (Table 13). The three volumes led to a FPF between 45-50%; however, the EF % was considerably lower at the 3.5 L total inspiratory volume (2.5 L ventilator volume plus volume applied through the device) compared to that at 4 and 4.5 L. This indicated that a higher volumetric flow was required for complete and efficient aerosolization of spray-dried powder.

Effect of Inspiration Wave Pattern

Spray-dried powder was again delivered through the catheter tube (3 mm ID) using similar conditions as before (60 L/min, 2 L of air). The ventilator delivered 2.5 L of air (20 L/min at 25% RH) using different inspiratory patterns; square, ramp and sine wave patterns. The FSI deposition profile indicated that the square and sine waves were superior to the ramp wave pattern (Table 13). The square wave inspiration pattern produced a FPF of ˜50% with an EF of ˜73% while the ramp led to a FPF of ˜32% and an EF of ˜74%. The sine wave achieved a deposition profile and performance closely resembling the square wave.

TABLE 13

| | | Aerosol parameters | | | |
|---|---|---|---|---|---|
| Different | | FPF % | FPD | | |
| ventilator settings | | (<5 um) | (<5 um, mg) | ED (mg) | EF (%) |
| Inspi- | 3.5 L | 48 ± 2.0 | 5.9 ± 0.4 | 12.3 ± 0.3 | 68 ± 1.0 |
| ratory | Square | | | | |
| Flow | wave | | | | |
| Volume | 4 L | 47 ± 2.5 | 7.0 ± 1.0 | 14.7 ± 1.0 | 80 ± 7.0 |
| | Square | | | | |
| | wave | | | | |
| | 4.5 L | 50 ± 5.0 | 6.6 ± 0.4 | 13.3 ± 0.3 | 73 ± 2.0 |
| Inspi- | Square | | | | |
| ration | wave | | | | |
| pattern | 4.5 L | 32 ± 3.0 | 4.3 ± 1.0 | 13.6 ± 1.0 | 74 ± 4.0 |
| | Ramp | | | | |
| | wave | | | | |
| | 4.5 L | 46 ± 4.0 | 6.7 ± 1.0 | 14.7 ± 1.0 | 81 ± 6.0 |
| | Sine | | | | |
| | wave | | | | |

FSI of spray-dried powder at different ventilator flow volume and inspiration pattern. (n = 3; ±S.D.).

Effect of Internal Diameter of Endotracheal Tube

The size and characteristics of the endotracheal tube influence aerosol deposition and play an important role in minimizing aerosol losses within artificial airways and increasing pulmonary deposition of drug in mechanically ventilated patients. In this set of experiments, the ventilator was set to deliver an inspiratory flow volume of 2.5 L through the endotracheal tube with a square wave inspiratory flow of 20 L/min (25% RH). The inhalation device was set to deliver the spray-dried drug at a flow rate of 60 L/min (2 L) using the 3 mm ID catheter tube. The two endotracheal tube internal diameters investigated were 6 mm and 8.5 mm. Increasing the tube diameter improved the FPF; however, EF was not affected (Table 14).

Effect of Internal Diameter of Catheter Tube

The standard configuration described for testing endotracheal tube diameter was also used to test different diameters of catheter tube. Three catheter tube internal diameters placed, within the 8.5 mm ID endotracheal tube, were investigated; 2.5 mm, 3 mm and 4 mm. Optimal aerosol delivery was obtained when the catheter extended the full length of the endotracheal tube. Increasing the catheter tube diameter from 2.5 mm to 4 mm decreased the resistance from 0.1537 KPa^{0.5}L⁻¹ min to 0.0695 KPa^{0.5}L⁻¹ min which in turn led to a significant improvement in the aerosol performance (Table 14). FPF decreased from ˜59% to ˜35% as the ID of the catheter decreased. The difference in ED for the different catheters was not statistically significant. Significant aerosol loss within the catheter tube was expected due to the large increase in the resistance as the ID decreased.

TABLE 14

| Different ID of | | Aerosol parameters | | | | |
|---|---|---|---|---|---|---|
| endotracheal and | | Resistance | FPF % | FPD mg | | |
| catheter tubes | | (KPa^{0.5} · L⁻¹ · min) | (<5 μm) | (<5 μm) | ED (mg) | EF (%) |
| Endotracheal | 6 mm | 0.119 | 40 ± 2.0 | 5.5 ± 0.1 | 13.8 ± 1.0 | 76 ± 4.0 |
| tube (3 mm ID | 8.5 mm | 0.102 | 50 ± 0.3 | 7.5 ± 0.3 | 14.8 ± 1.0 | 81 ± 300 |
| catheter tube) | | | | | | |
| Catheter tube | 2.5 mm | 0.154 | 35 ± 3.0 | 4.6 ± 0.4 | 13.2 ± 0.4 | 73 ± 2.0 |
| (8.5 mm ID | 3 mm | 0.102 | 50 ± 0.3 | 7.5 ± 0.3 | 14.8 ± 1.0 | 81 ± 3.0 |
| endotracheal | 4 mm | 0.070 | 59 ± 4.0 | 8.0 ± 1.0 | 13.6 ± 0.3 | 75 ± 1.0 |
| tube) | | | | | | |

FSI of spray-dried powder using different internal diameters (id) of endotracheal and catheter tubes. (n = 3; ±S.D.).

Effect of Delivery Mass of Spray-Dried Powder

The standard configuration, described immediately above, was used and spray-dried powders were delivered in three different masses (20 mg, 40 mg and 80 mg) using either 3 mm or 4 mm ID catheter tubes (Table 15). For both 3 mm and 4 mm ID catheter tubes, EF was relatively unaffected by the increase in the delivery mass. The FPF generally decreased when the delivery mass was increased from 20 mg to 80 mg. Both EF and FPF were higher when the ID of the catheter tube was increased from 3 mm to 4 mm for the corresponding delivery mass. For a delivery mass of 80 mg, a FPD of ˜29±1 mg was achieved using the 4 mm catheter tube (Table 15).

TABLE 15

| | | Aerosol parameters | | | |
|---|---|---|---|---|---|
| | | FPF % | FPD | | |
| Delivery mass (mg) | | (<5 um) | (<5 um, mg) | ED (mg) | EF (%) |
| 3 mm ID | 20 mg | 50 ± 5.0 | 6.6 ± 0.4 | 13.3 ± 0.3 | 73 ± 2.0 |
| Catheter | 40 mg | 45 ± 4.0 | 12.6 ± 1.0 | 28.2 ± 2 | 78 ± 5.0 |
| tube | 80 mg | 43 ± 2.0 | 26.8 ± 0.4 | 62.5 ± 2.0 | 87 ± 3.0 |
| 4 mm ID | 20 mg | 59 ± 4.0 | 8.0 ± 1.0 | 13.6 ± 0.3 | 75 ± 1.0 |
| Catheter | 40 mg | 58 ± 3.0 | 15.5 ± 0.2 | 27 ± 1.0 | 74 ± 3.0 |
| tube | 80 mg | 51 ± 2.0 | 28.7 ± 1.0 | 56.7 ± 3.0 | 78 ± 4.0 |

FSI of spray-dried powder containing various doses using different internal diameters (ID) of catheter tube. (n = 3; ±S.D.).

Effect of Humidity

The effect of humidity on the aerosol performance was investigated by applying three different relative humidity settings (25% RH, 50% RH and 75% RH) through the ventilator and endotracheal tube (Table 16). The standard configuration was used again. As indicated by the FSI profile, the increase in relative humidity decreased aerosol performance as expected. Even though the EF was unaffected by the increase in relative humidity, FPF decreased from ˜50% to ˜28% when relative humidity was increased from 25% to 75%, respectively. The decrease was more pronounced from 25% to 50% as compared to 50% to 75%. The reason for this decrease in FPF was likely due to an increase in agglomeration of drug particles with increasing humidity. This phenomenon is usually more pronounced if the drug particle is hygroscopic.

TABLE 16

| Aerosol parameters | 25% RH | 50% RH | 75% RH |
|---|---|---|---|
| FPF % (<5 um) | 50 ± 5.0 | 32 ± 2.0 | 28 ± 2.0 |
| FPD (<5 um, mg) | 6.6 ± 0.4 | 4.1 ± 0.5 | 4.0 ± 0.4 |
| ED (mg) | 13.3 ± 0.3 | 13.0 ± 1.0 | 14.3 ± 1.0 |
| EF (%) | 73 ± 2.0 | 72 ± 4.0 | 79 ± 6.0 |

FSI of spray-dried powder under different humidity conditions. (n = 3; ±S.D.).

Comparison of Different Dry Powder Formulations.

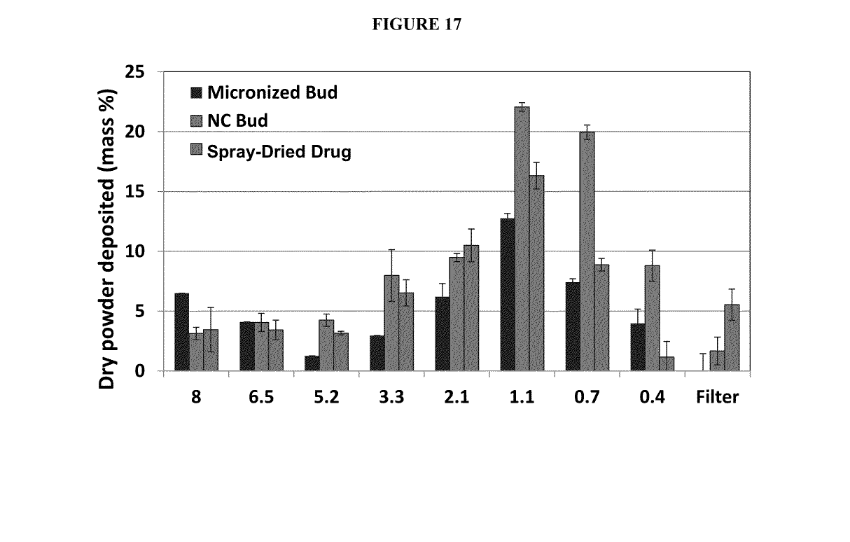

Dry powder formulations were prepared and delivered from a Monodose inhaler according to FIG. 4A and formulation characteristics were determined as described above. As seen in Table 17, the % EF for micronized budesonide is below the threshold for suitable dry powder formulations for lung delivery according to the present disclosure. FIG. 17 is a plot showing the above dry powder formulations analyzed using ACI.

TABLE 17

| Characteristics | | Micronized | | Spray-dried |
|---|---|---|---|---|
| of dry powder | | Bud | NC-Bud | drug |
| Fill mass (mg) | | 3.2 ± 0.1 | 2.95 ± 0.1 | 30.05 ± 1 |
| % EF^{a} | | 49 ± 1 | 88 ± 3 | 80 ± 1 |
| % FPF^{b} | ≦5 μm | 74 ± 2 | 79 ± 3 | 73 ± 4 |
| | ≦3 μm | 65 ± 3 | 68 ± 2 | 59 ± 6 |

| | | | |
|---|---|---|---|
| % Delivery Efficiency <5 μm | 37 ± 0.4 | 70 ± 5 | 58 ± 6 |
| MMAD^{c} | 2 ± 0.2 | 1.5 ± 0.1 | 1.92 ± 0.1 |
| GSD^{d} | 2.92 ± 0.2 | 2.5 ± 0.1 | 2.6 ± 0.2 |
| ED^{e} (mg) | 1.54 ± 0.01 | 2.6 ± 0.03 | 20.03 ± 1 |

^{a}% EF = Percent emitted fraction.
^{b}FPF = Fine particle fraction.
^{c}MMAD = Mass median aerodynamic diameter obtained from cascade impactor.
^{d}GSD = Geometric standard deviation.
^{e}ED = Emitted Dose
Cascade impaction results of different dry powder therapeutic formulations at a flow rate of 90 L/min for 2.6 s (values = average ± S.D., n = 3).

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. An system comprising:
an air source;
a inhalation device operably connected to the air source; and
a dry powder therapeutic formulation disposed within the inhalation device, the dry powder therapeutic formulation having an emitted fraction greater than 60% as measured by the emitted fraction test.

2. The system of claim 1 wherein the air source is a ventilator or a positive pressure pump.

3. The system of claim 1 wherein the air source includes a ventilator and a positive pressure pump and wherein the system further comprises a valve for regulating the flow of air.

4. The system of claim 1 further comprising a catheter tube capable of insertion into a ventilator circuit.

5. The system of claim 1 further comprising a catheter tube capable of insertion into a ventilator circuit disposed proximate to the inhalation device.

6. The system of claim 1 further comprising a tracheal tube.

7. The system of claim 1 further comprising a tracheal tube comprising an inflation cuff.

8. The system of claim 1, wherein inhalation device comprises:
a body adapted for connection with an inlet tube at one end, which comprises a cone-shaped chamber having a passageway for the movement of air there through and a receptacle configured to receive a medicament container; and
a cap adapted for connection with an outlet tube at one end and for receiving the body at the other end.

9. The system of claim 1, wherein the dry powder therapeutic formulation has a mass mean aerodynamic diameter less than 3.5 μm.

10. An inhalation device comprising:
a body adapted for connection with an inlet tube at one end, which comprises a cone-shaped chamber having a passageway for the movement of air therethrough and a receptacle configured to receive a medicament container; and
a cap adapted for connection with an outlet tube at one end and for receiving the body at the other end.

11. A method comprising delivering into a subject's lungs a dry powder therapeutic formulation having an emitted fraction greater than 60% as measured by the emitted fraction test.

12. The method of claim 11, wherein the dry powder therapeutic formulation has a mass mean aerodynamic diameter less than 3.5 μm.

13. The method of claim 11, wherein the dry powder therapeutic formulation is introduced into a ventilation circuit.

14. The method of claim 11, wherein the dry powder therapeutic formulation is introduced into a catheter.

15. The method of claim 11, wherein the dry powder therapeutic formulation is initially disposed within an inhalation device according to claim 10.
